(19) United States
(12) Patent Application Publication
(10) Pub. No.: US 2022/0090167 A1
(43) Pub. Date: Mar. 24, 2022

(54) ASYMMETRIC NANOPORE MEMBRANE (ANM) FILTRATION FOR HIGH-EFFICIENCY VIRUS ENRICHMENT AND PURIFICATION

(71) Applicant: University of Notre Dame du Lac, Notre Dame, IN (US)

(72) Inventors: Ceming Wang, South Bend, IN (US); Hsueh-Chia Chang, South Bend, IN (US)

(21) Appl. No.: 17/474,296

(22) Filed: Sep. 14, 2021

Related U.S. Application Data
(60) Provisional application No. 63/078,533, filed on Sep. 15, 2020.

(51) Int. Cl. C12Q 1/6806; C12N 7/00; C12Q 1/70

(52) U.S. Cl. CPC C12Q 1/6806; C12N 7/00; C12N 2770/18051; C12N 2740/15051; C12Q 1/70

(57) ABSTRACT

Described herein is a method for high-efficiency virus enrichment and purification using an asymmetric nanopore membrane (ANM) filtration technology. The ANM design prevents viral particle deformation, lysing, and fusion due to the strong external force and thus significant increases the yield while preserving other advantages of size-based ultrafiltration. It also offers a unique feature of being able to flush the contaminating proteins from the viral particles. It offers higher throughput, yield, sample purity, concentration factor, and more precise size fractionation than current approaches.

### CROSS-REFERENCE TO RELATED APPLICATIONS

This application claims priority to U.S. Provisional Patent Application No. 63/078,533, filed on Sep. 15, 2020, which is incorporated by reference herein in its entirety.

### REFERENCE TO SEQUENCE LISTING

This application is filed with a Computer Readable Form of a Sequence Listing in accord with 37 C.F.R. § 1.821(c). The text file submitted by EFS, “092012-9140-US02_sequence_listing_18-AUG-2021_ST25.txt” was created on Aug. 18, 2021, contains 7 sequences, has a file size of 40.1 Kbytes, and is hereby incorporated by reference in its entirety.

### FEDERALLY SPONSORED RESEARCH

This invention was made with United States government support under National Institutes of Health grant numbers 1R21CA206904-01 and HG009010-01. The United States government has certain rights in the invention.

### TECHNICAL FIELD

Described herein is a method for high-efficiency virus enrichment and purification using an asymmetric nanopore membrane (ANM) filtration technology. The ANM design prevents viral particle deformation, lysing, and fusion due to the strong external force and thus significant increases the yield while preserving other advantages of size-based ultrafiltration. It also offers a unique feature of being able to flush the contaminating proteins from the viral particles. It offers higher throughput, yield, sample purity, concentration factor, and more precise size fractionation than current approaches.

### BACKGROUND

Nucleic acid amplification-based tests currently offer the most sensitive and early detection of COVID-19. Nucleic acid tests are being used in the ongoing coronavirus pandemic as an essential tool to track the spread of the disease. However, it has been found that the chance of a false negative result is greater than 21% and, at times, far higher [1]. Over the 4 days of infection before the typical time of symptom onset (day 5), the probability of a false negative result in an infected person decreases from 100% on day 1 to 68% on day 4. On the day of symptom onset, the median false-negative rate remains high at 38%. Even worse, there is accumulating evidence suggesting that transmission from persons who are presymptomatic (SARS-CoV-2 detected before symptom onset) or asymptomatic (SARS-CoV-2 detected but symptoms never develop) [2]. The possible high false-negative rate in these cases pose a major challenge to current intervention measures including widespread testing and contact tracing to detect asymptomatic infections, interrupt undetected transmission chains, and further bend the curve downward. Therefore, there is an urgent need to increase the sensitivity of current RT-PCR COVID-19 tests.

The high false-negative rate can be decreased by improving the Limit of Detection (LOD) of COVID-19 Tests. The LOD of current FDA-approved COVID-19 tests are still relatively high (e.g., LabCorp: 6,250 copies/mL; CDC: 1000-3000 copies/mL) [3]. Given that the RT-PCR reaction was shown to be very sensitive for accurately detecting viral genomes present in a sample (down to just 1-10 molecules of RNA) [4], the high LOD of current COVID-19 tests are mainly due to the significant target loss associated with current sample preparation steps commonly used for RT-PCR tests. FIG. 1 shows a typical workflow for current COVID-19 RT-PCR testing. Usually, a clinician collects a nasopharyngeal swab and transfers it to a vial containing a few milliliters (typically 1.5-3 mL, minimum volume for swab soaking) of viral transport medium (VTM), which is transported to a laboratory for testing. The viral RNA is then purified from only a fraction of the swab VTM sample (typically 100 μL, 1/30th of the swab) using column-based RNA purification kits, leading to a 96.6% target loss. Moreover, a small fraction (5 μL) of the eluted purified RNA (100 μL) is then reverse transcribed and amplified, corresponding to another 95% RNA loss. As a result, only about 0.16% of the viral RNA from the swab is extracted for RT-PCR even if the yield of the RNA extraction kit is assumed to be 100%. According to a recent study [5], patient viral titers are high during the first days of infection and a single patient nasopharyngeal swab may harbor close to 1 million SARS-COV-2 viral particles. This means that no more than about 1,600 RNA molecules are available for RT-PCR quantification using the current gold-standard sample preparation method. In fact, patient viral titers vary a lot and can be orders of magnitude lower, inevitably resulting in a high false negative rate. Thus, there is a need for a straightforward way to reduce the target loss, such as concentrating the virus before subjecting the sample to RT-PCR.

### SUMMARY

One embodiment described herein is a system for isolating viral particles comprising: a first chamber; a second chamber; a membrane positioned between the first and second chambers, and comprising a first membrane surface facing and at least partially defining the first chamber, a second membrane surface facing and at least partially defining the second chamber and a plurality of asymmetrically shaped nanopores extending between the first and second membrane surfaces, wherein each nanopore includes a first nanopore opening at the first membrane surface having a first diameter, and a second nanopore opening at the second membrane surface having a second diameter that is greater than the first diameter; a sample comprising the viral particles positioned within the first chamber; and a device for inducing fluid flow through the membrane from the first chamber to the second chamber by pressure driven flow, electroosmotic flow, centrifugal force, or a combination thereof, In one aspect, the first membrane surface comprises one or more baffles.

Another embodiment described herein is a system for isolating viral particles comprising: a first chamber; a second chamber; a membrane positioned between the first and second chambers, and comprising a first membrane surface facing and at least partially defining the first chamber, a second membrane surface facing and at least partially defining the second chamber and a plurality of asymmetrically shaped nanopores extending between the first and second membrane surfaces, wherein each nanopore includes a first nanopore opening at the first membrane surface having a first diameter, and a second nanopore opening at the second membrane surface having a second diameter that is greater than the first diameter; wherein the first membrane surface comprises one or more baffles; a sample comprising the viral particles positioned within the first chamber; and a device for inducing fluid flow through the membrane from the first chamber to the second chamber by pressure driven flow, electroosmotic flow, centrifugal force, or a combination thereof. In one aspect, the first membrane surface is coated with a magnetic alloy. In another aspect, the first diameter is from about 10 nm to about 200 nm. In another aspect, the first diameter of the plurality of asymmetrically shaped nanopores has a coefficient of variation of less than 10% between each nanopore. In another aspect, the second diameter is from about 30 nm to about 10 μm. In another aspect, a distance between the first and second membrane surfaces is from about 1 μm to about 100 μm. In another aspect, the membrane comprises a nanopore density from about 10⁶ to about 10¹⁰ nanopores/cm². In another aspect, the nanopores of the membrane are ion-etched. In another aspect, the first chamber comprises a plurality of inlets. In another aspect, the first chamber comprises a first inlet for loading of the sample into the first chamber; and, a second inlet for loading of an elution buffer, lysing solution, PCR cocktail, or a combination thereof into the first chamber; and, wherein a concentrated virus solution is eluted from the first chamber through the first inlet or the second inlet into a collection tube or a third chamber. In another aspect, the first inlet and second inlet are the same inlet. In another aspect, the second chamber comprises an outlet wherein the device for inducing fluid flow through the membrane from the first chamber to the second chamber is connected. In another aspect, the membrane is formed from one or more materials comprising one or more of a polyethylene terephthalate (PET), a polycarbonate (PC), a polypropylene (PP), a polyimides (PI), or a polyethersulphone (PES). In another aspect, the system as described herein further comprises a fourth chamber and a filter positioned between the fourth chamber and the first chamber, the filter comprising a first filter surface facing and at least partially defining the fourth chamber, a second filter surface facing and at least partially defining the first chamber and a plurality of filter pores extending between the first and second filter surfaces. In another aspect, each filter pore has a diameter of about 200 nm to about 5 microns. In another aspect, the filter is formed from one or more materials comprising a polyethylene terephthalate (PET), a polycarbonate (PC), a polypropylene (PP), a polyimides (PI), and a polyethersulphone (PES). In another aspect, the device for inducing fluid flow generates a flow rate of about 0.01 mL/hour to about 100 ml/hour. In another aspect, the device for inducing fluid flow generates a pressure less than about 1 atm. In another aspect, the device for inducing fluid flow comprises a syringe pump, an electroosmotic pump, a micropump, a centrifuge, a vacutainer, a snap lock syringe pump, or a combination thereof, In another aspect, the sample is applied perpendicularly or tangentially to the membrane or the filter. In another aspect, when the sample is applied tangentially to the membrane or filter, a flow rate of about 5 mL/hour to about 40 mL/hour. In another aspect, the viral particles are about 80-100 nm in size. In another aspect, the viral particles are SARS-COV-2 viral particles. In another aspect, the magnetic alloy is nickel-iron, samarium-cobalt, aluminum-nickel-cobalt, nickel-iron-chromium, iron-chromium-cobalt, or neodymium-iron-boron. In another aspect, the viral particles are bound to a probe that is coupled to a magnetic bead. In another aspect, the probe is an antibody. In another aspect, the system is connected with a plurality of identical systems in series or in parallel.

Another embodiment described herein is a use of the system described herein for isolating a virus.

Another embodiment described herein is a method for isolating viral particles comprising: providing the system as described herein and inducing fluid flow through the membrane from the first chamber to the second chamber, whereupon the viral particles are isolated in the second chamber.

Another embodiment described herein is a viral particle isolated using a method described herein.

Another embodiment described herein is a method for detecting viral particles in a sample comprising: providing a system described herein; inducing fluid flow through the membrane from the first chamber to the second chamber, whereupon the viral particles are isolated in the second chamber; lysing the isolated viral particles; and measuring viral RNA. In one aspect, the isolated viral particles are lysed using chemical, mechanical, or thermal lysing. In another aspect, when chemical lysing is used, RNA extraction is performed on the isolated viral particles before the viral RNA is measured. In another aspect, when thermal or mechanical lysing is used, the viral RNA is directly measured. In another aspect, the lysed viral particles are mixed with a PCR cocktail in the first chamber. In another aspect, the sample has an initial volume of about 1 mL to about 100 mL. In another aspect, the sample is collected by a swab. In another aspect, the sample is extracted from the swab in a buffer. In another aspect, the sample comprising the viral particles comprises one or more of cell culture supernatants or a sample obtained from an animal subject. In another aspect, the sample obtained from an animal subject comprises one or more of blood, saliva, droplets from coughing, droplets from sneezing, plasma, tear, serum, urine, sputum, pleural effusion, or ascites.

### DESCRIPTION OF THE DRAWINGS

FIG. 1 shows a representative workflow for the current COVID-19 RT-PCR test according to the CDC 2019-Novel Coronavirus (2019-nCoV) Real-Time RT-PCR Diagnostic Panel instruction. Mainstream kits and RT-PCR reagent are listed for illustration.

FIG. 2 shows a schematic summary of the viral particle sample preparation, and downstream RT-PCR detection. The size of lentivirus is about 80-100 nm, similar to SARS-CoV-2. Thermal lysing was performed at 75° C. for 10 min.

FIG. 3 shows the ANM setup.

FIG. 4A-C show schematics of the ANM virus enrichment and isolation device. FIG. 4A shows the schematic overview of the ANM virus enrichment and isolation device. FIG. 4B shows the workflow of the ANM virus enrichment and isolation device. The proposed procedure for viral RNA extraction involves: concentration and isolation of viral particles such as SARS-CoV-2 on the surface of ANM; and lysing of the captured viral particles using 1% Triton X-100 and elution of released viral RNA for direct RT-PCR. FIG. 4C shows SEM images of the ANM.

FIG. 5A-B show the detection of lentiviruses with ANM concentration compared to the standard procedure. FIG. 5A shows the Cₜ of samples with and without ANM. FIG. 5B shows the Ct of sample flow through with and without ANM.

FIG. 6A-B show the detection of lentiviruses with direct RT-PCR using ANM concentration. FIG. 6A shows the Cₜ of chemically and thermally lysed of samples. FIG. 6B shows the Cₜ of samples using direct RT-PCR with and without ANM. FIG. 6C shows the Cₜ of 2.5 and 10 mL samples with and without ANM,

FIG. 7 shows a comparison of the typical processing time between ANM and conventional track-etched membranes with the same pore size (˜60 nm) when 2.5 mL viral transport medium (VTM) sample is processed. The filtration step was driven by a very low negative pressure (˜0.8 atm) of a vacuum tube produced by a syringe.

FIG. 8A-B shows Cₜ values from RT-PCR performed with the same SARS-CoV-2 sample before and after using two different concentration devices: ANM device and commercial ultrafiltration device (Amicon Ultra-2 Centrifugal Filter Unit from Millipore, UFC210024). These experiments are shown for two different primer-probe sets: N1 gene (FIG. 8A) and N2 gene (FIG. 88), For all concentration experiments, the elution volume was 0.2 mL with an input sample volume of 1 mL. Two different lysing methods (an RNA extraction kit and surfactant-based lysing method using 1% (vol./vol.) Triton X-100) were used for comparison.

FIG. 9A-B show Cₜ values from RT-PCR performed on the same SARS-CoV-2 sample without concentration using three different lysing methods: an RNA extraction kit, thermal lysing (65° C., 10 min), and surfactant-based lysing using a different percent (vol./vol.) of Triton X-100. These experiments are shown for two different primer-probe sets: N1 gene (FIG. 9A) and N2 gene (FIG. 9B). The lysing performance of Triton X-100 was comparable with the RNA extraction kit and thermal lysing (65° C., 10 min).

FIG. 10A-B show Cₜ values from RT-PCR performed on the same SARS-CoV-2 sample before and after ANM enrichment from different input volumes (1 mL, 2.5 mL, and 5 mL). These experiments are shown for two different primer-probe sets: N1 gene (FIG. 10A) and N2 gene (FIG. 10B). The ANM devices concentrated the virus samples from various volumes to a final elution volume of 40 μL. 1% Triton X-100 was used to lyse the viral particles for direct RT-PCR. These data show that the ANM devices enrich viral particles from large volume samples and thus boost the downstream assay sensitivity.

FIG. 11A-B show Cₜ values from RT-PCR performed on SARS-CoV-2 samples with different viral loads before and after ANM enrichment. These experiments are shown for two different primer-probe sets: N1 (FIG. 11A) and N2 gene (FIG. 11B). The ANM devices concentrated the virus samples from 2.5 mL to a final elution volume of 40 μL. 1% Triton X-100 was used to lyse the viral particles for direct RT-PCR. These data show that the ANM devices enrich viral particles even in samples with a very low viral titer and thus boost the downstream assay sensitivity by eliminating false negatives.

FIG. 12A-B show the tangential flow ANM filtration devices in series. Each chip consists of an ANM membrane at the bottom and a baffled substrate at the top. The concentrated virus solution is pumped tangentially between the two substrates of the chip. The tangential flow design and the baffle prevent the buildup of a filter cake of virus that would reduce permeate flow.

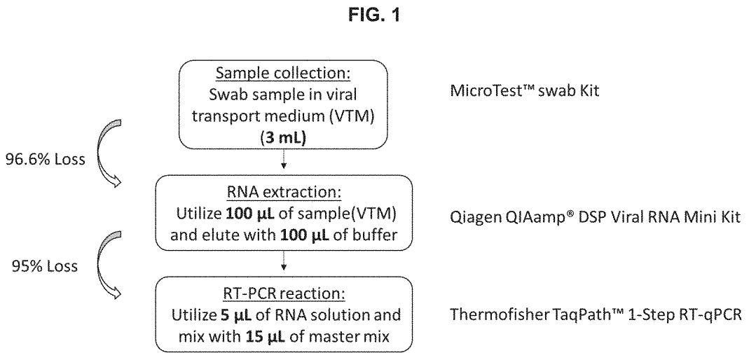

### DETAILED DESCRIPTION

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are those that are well known and commonly used in the art. In case of conflict, the present document, including definitions, will control. Preferred methods and materials are described below, although methods and materials similar or equivalent to those described herein can be used in practice or testing of the present invention.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified. As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more than 3 standard deviations, per the practice in the art, Alternatively, such as with respect to biological systems or processes, the term “about” can mean within an order of magnitude, in some embodiments within 5-fold, and in some embodiments within 2-fold, of a value.

All ranges disclosed herein include both end points as discrete values as well as all integers and fractions specified within the ranges. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points. As used herein, the symbol “˜” means “about.”

Coronaviruses (CoVs), are enveloped positive-sense RNA viruses, which are surrounded by crown-shaped, club-like spikes projection on the outer surface. Coronaviruses' spike proteins are glycoproteins that are embedded over the viral envelope. This spike protein attaches to specific cellular receptors and initiates structural changes of spike protein, and causes penetration of cell membranes, which results in the release of the viral nucleocapsid into the cell. These spike proteins determine host trophism. Coronaviruses have a large RNA genome, ranging in size from 26 to 32 kilobases and capable of obtaining distinct ways of replication. Like other RNA viruses, coronaviruses under-go replication of the genome and transcription of mRNAs upon infection. Coronavirus infection in a subject can result in significant and long-term damage of the lungs, leading to possibly sever respiratory issues.

As used herein “BARS-COV-2” is a betacoronavirus (Beta-CoV or β-CoV). In particular, SARS-COV-2 is a Beta-CoV of lineage B. SARS-COV-2 may also be known as 2019-nCoV, COVID-2019 or 2019 novel coronavirus. Betacoronaviruses are one of four genera of coronaviruses and are enveloped, positive-sense, single-stranded RNA viruses of zoonotic origin, Betacoronaviruses mainly infect bats, but they also infect other species like humans, camels, and rabbits. SARS-COV-2 may be transferable between animals, such as between humans. Beta-CoVs may induce fever and respiratory symptoms in humans. The overall structure of S-CoV genome contains an ORF1ab replicase polyprotein (rep, pp1ab) preceding other elements. This polyprotein is cleaved into many nonstructural proteins. SARS-COV-2 has a phenylalanine in the (F486) in the flexible loop of the receptor binding domain, flexible glycyl residues, and a four amino acid insertion at the boundary between the S1 and S2 subunits that results in the introduction of a furin cleavage site. The furin cleavage site may result in SARS-COV-2 tissue tropism, increase transmissibility, and alter pathogenicity.

As used herein, “sample” can mean any sample in which the presence and/or level of a target is to be detected or determined or any sample comprising a viral particle, or component thereof as described herein. Samples may include liquids, solutions, emulsions, or suspensions. Samples may include any plant fluid or tissue, such as apoplastic fluid, any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, pleural effusion, ascites, digestive fluid, skin, or combinations thereof. The sample can be used directly as obtained from a subject or can be pre-treated, such as by filtration, distillation, extraction, concentration, centrifugation, inactivation of interfering components, addition of reagents, and the like, to modify the character of the sample in some manner as discussed herein or otherwise as is known in the art.

As used herein, the term “subject” refers to an animal. Typically, the animal is a mammal. A subject also refers to, for example, primates (e.g., humans, male or female; infant, adolescent, or adult), pigs, cows, sheep, goats, horses, dogs. cats, rabbits, rats, mice, fish, birds, and the like. In one embodiment, the subject is a human.

“Virus,” “virus particles,” and “viral particles” are used interchangeably herein. Viruses may be considered nanoparticles. Virus as used herein can be a particle that can infect a cell of a biological organism. An individual virus, or a virus particle, also can be called a virion, can comprise one or more nucleic acid molecules, so called viral genome, surrounded by a protective protein coat known as a capsid. Unlike cellular organisms, in which the nucleic acid molecules are generally made up of DNA, the viral nucleic acid molecule may comprise either DNA or RNA. In some cases, viral nuclear acid molecules comprise both DNA and RNA. Viral DNA is usually double-stranded, either a circular or a linear arrangement, while viral RNA is usually single-stranded. However, examples of single stranded viral DNA and double-stranded viral RNA are also known. Viral RNA may be either segmented (with different genes on different RNA molecules) or nonsegmented (with all genes on a single piece of RNA). The size of the viral genome can vary significantly in size. Both DNA and RNA viruses can be isolated herein.

Described herein are methods for high-efficiency virus enrichment and purification using an asymmetric nanopore membrane (ANM) filtration technology. The ANM technology utilizes an asymmetric etching technique for commercial ion-track membranes to produce conic nanopores that can range from 10 nm to 200 nm on the tip side and up to 2 microns on the base side. Track-etched membranes that have asymmetrically shaped pores (as opposed to the more conventional cylindrical or irregularly shaped pores in ultrafiltration membranes) offer an important advantage for viral isolation applications. The key advantage of the symmetrical pore shape is a dramatic 200-400% reduction in the applied pressure/force to drive the sample through the filter membrane at the same throughput, compared to an analogous cylindrical pore membrane. This significant reduction in applied pressure avoids lysing of viruses due to high pressure while preserving other advantages of size-based ultrafiltration. Moreover, the chance of dogging and trapping is significantly reduced due to a dramatic enhancement in the rate of transport through the membrane, relative to an analogous cylindrical pore membrane. This new pore geometry design allows high yield and high throughput and permits trapping designs. The trapping design allows for concentration of viruses within a specific size range and separation from the larger and smaller debris, molecules, and viruses. The concentration factor can be as large as 100. Importantly, the trapping design allows for flushing of the trapped viruses with rinsing buffer to remove all contaminants, including the abundant proteins. It also offers higher throughput, yield, sample purity and concentration factor than current products, plus more precise size fractionation.

AMN technology allows for precise control of the pore size such that size-based fractionation can be performed within the 30-200 nm range (by using different nanopore membrane modules with different pore sizes).

ANM consists of a membrane holder and a commercial micropump or syringe pump. The pump can be housed in a dedicated instrument or the consumers can use their own syringe pumps in their laboratories. One embodiment includes the ANM and its holder, which may be disposed after each use. The ANM may be fabricated from the polycarbonate track-etched membranes, which are initially irradiated to create the desired ion tracks and then etched to develop tracks into pores. The track irradiation step is capable of mass production. The etching process involves chemical etching and dry etching, which are also easy to scale up.

The ANM is high throughput, as the conic geometry reduces the flow shear rate. The lower shear rate also minimizes virus loss due to lysing. The result is a high-yield and high-throughput platform that can isolate viruses from other nanoparticles such as proteins, RNPs, HDL, and LDL. The conic nanopore is fabricated by asymmetric wet etching of ion-track membranes without dielectric coating. The technology has been validated with cell medial supernatant and plasma samples. ANM exhibits a much higher yield and throughput than precipitation technology (Exoquick), ultracentrifugation, size-exclusion (qEV), and column adsorption (miReasy). The throughput is particularly high, taking about 1 hour for about 1 mL cell media and about 300 microliter plasma, compared to days for the other technologies. qEV has a comparable throughput but it does not fractionate.

The isolated and purified virus particles can be lysed mechanically, thermally, or chemically to release their molecular biomarker cargo for quantification. Such quantification can be done with many technologies, including real-time quantitative PCR (qRT-PCR), one-step qRT-PCR, and ANM miRNA quantification technology that does not suffer from PCR-amplification bias. The AMN filtration technology allows for complete virus particle and protein separation due to the presence of the 60 nm asymmetric nanopore filter and the addition buffer washing step for the trapped virus particles between the two membranes. Thus, high recovery efficiency can be achieved without sacrificing protein removal. Additionally, this method doesn't require timing which introduces significant complexity in the isolation process and reduces throughput. The ANM technology isolates and concentrates virus particles at the same time from any arbitrary volume up to 10 mL, up to 5 mL, up to 4 mL, up to 3 mL, up to 2 mL, up to 1 mL, up to 500 μL, or up to 300 μL. The concentration factor can be as large as a factor of 10 to 100. The present nanopore technology allows the same isolation efficiency for all virus particles with a size larger than the tip size of the pore, thus less bias is introduced in the isolation step. AMN technology allows for precise control of the pore size such that size-based fractionation can be performed within the 30-200 nm range (by using different nanopore membrane modules with different pore sizes).

One embodiment described herein is a system for isolating viral particles comprising: a first chamber; a second chamber; a membrane positioned between the first and second chambers, and comprising a first membrane surface facing and at least partially defining the first chamber, a second membrane surface facing and at least partially defining the second chamber and a plurality of asymmetrically shaped nanopores extending between the first and second membrane surfaces, wherein each nanopore includes a first nanopore opening at the first membrane surface having a first diameter, and a second nanopore opening at the second membrane surface having a second diameter that is greater than the first diameter; a sample comprising viral particles positioned within the first chamber; and a device for inducing fluid flow through the membrane from the first chamber to the second chamber by pressure driven flow, electroosmotic flow, centrifugal force, or a combination thereof. In one aspect, the first chamber comprises a wall opposite of the first membrane that comprises one or more baffles. In some embodiments, there may be at least 1, at least 2, at least 3, at least 4, at least 5 baffles. In other embodiments, there may be at most 1000, at most 900, at most 800, at most 700, at most 600, at most 500, at most 400, at most 300, at most 200, at most 100, at most 50, or at most 25 baffles. The baffles may be made of fiberglass, plastic, a composite, or another material. In some embodiments, the baffles may be made of polycarbonate (PC), polystyrene (PS), polyethylene terephthalate (PET), polyvinylchloride (PVC), SU-8 photoresist and polyimide (PI), polydimethylsiloxane (PDMS), silicon, or glass. In a particular embodiment, the baffles may be made of polymethyl methacrylate (PMMA). The baffles can be shaped like cubes, triangular prisms, rectangles, cones, or panels that are curved, zigzagged, corrugated or L-shaped, have a combination of these shapes, or are otherwise configured. The baffle geometry can be triangle, wedge, crescent etc. They can assume regimented or staggered patterns, including herringbone patterns. In a particular embodiment, the baffles may be cubes or triangular prisms. The baffles can have a height ranging from about 15 μm to about 3 mm, about 20 μm to about 2 mm, about 25 μm to about 2 mm, about 30 μm to about 2 mm, about 35 μm to about 1 mm, about 40 μm to about 1 mm, or about 45 μm to about 1 mm. The baffles may be spaced from about 25 μm to about 7 mm, about 50 μm to about 6 mm, about 100 μm to about 5 mm, about 100 μm to about 4 mm, about 100 μm to about 3 mm, about 100 μm to about 2 mm, about 100 μm to about 1 mm, about 125 μm to about 5 mm, or about 150 μm to about 5 mm apart. The size, number, and spacing of the baffles may vary and be selected to provide the sample flow dispersion, route, and rate desired for a particular use or particle to be isolated. In some embodiments, each or particular baffles have gaps formed at both the top and/or the bottom, at one or both sides, all the way around them. In addition, the baffles may be arranged in an array with a regular pattern or an irregular arrangement. And some of the baffles may be larger than other ones. Previously ultrafiltration baffles have been placed directly on a membrane to produce vortices that break up filter cakes. The vortices, however, will also reduce the filtration rate. The present disclosure places the baffles on the channel surface opposite of the membrane without producing vortices. The arrangement and spacing of the baffles depends on various factors such as the size range of the viral particles or nanoparticles, diffusivity in that particular medium, membrane thickness, etc. and can be dictated through the diffusion timescale of the polarized layer, the normal and tangential flow rates, and the entrance length of the fluid flow. The baffles produce an upward lift to disrupt the filter cake before it is well packed.

Another embodiment described herein is a system for isolating viral particles comprising: a first chamber; a second chamber; a membrane positioned between the first and second chambers, and comprising a first membrane surface facing and at least partially defining the first chamber, a second membrane surface facing and at least partially defining the second chamber and a plurality of asymmetrically shaped nanopores extending between the first and second membrane surfaces, wherein each nanopore includes a first nanopore opening at the first membrane surface having a first diameter, and a second nanopore opening at the second membrane surface having a second diameter that is greater than the first diameter; wherein the first chamber comprises a wall opposite of the first membrane that comprises one or more baffles; a sample comprising viral particles positioned within the first chamber; and a device for inducing fluid flow through the membrane from the first chamber to the second chamber by pressure driven flow, electroosmotic flow, centrifugal force, or a combination thereof. In one aspect, the first membrane surface may be coated with a magnetic alloy. In another aspect, the system may further comprise a fourth chamber and a second membrane positioned between the fourth chamber and the second chamber, comprising a first membrane surface facing and at least partially defining the second chamber and a second membrane surface facing and at least partially defining the fourth chamber. The second membrane may be the membrane as described herein (e.g., ANN) and the first membrane surface of the membrane may be coated with a magnetic alloy. In another aspect, the magnetic alloy is nickel-iron, samarium-cobalt, aluminum-nickel-cobalt, nickel-iron-chromium, iron-chromium-cobalt, or neodymium-iron-boron. In another aspect, the viral particles are bound to a probe that is coupled to a magnetic bead. The magnetic bead may be any of a wide variety of shapes, such as spherical, generally spherical, egg shaped, disc shaped, cubical and other three-dimensional shapes. The magnetic beads may be manufactured using a wide variety of materials, including for example, resins, and polymers. The magnetic beads may be any suitable size, including for example, microbeads, microparticles, nanobeads and nanoparticles. The magnetic beads may comprise a magnetically responsive material that may constitute substantially all of a bead or one component only of a bead. The remainder of the bead may include, among other things, polymeric material, coatings, and moieties which permit attachment of an assay reagent. Examples of suitable magnetic beads include flow cytometry microbeads, polystyrene microparticles and nanoparticles, functionalized polystyrene microparticles and nanoparticles, coated polystyrene microparticles and nanoparticles, silica microbeads, fluorescent microspheres and nanospheres, functionalized fluorescent microspheres and nanospheres, coated fluorescent microspheres and nanospheres, color dyed microparticles and nanoparticles, magnetic microparticles and nanoparticles, superparamagnetic microparticles and nanoparticles (e.g., DYNABEADS® particles, available from Dynal Bead Based Separations (Invitrogen Group), Carlsbad, Calif.), fluorescent microparticles and nanoparticles, coated magnetic microparticles and nanoparticles, ferromagnetic microparticles and nanoparticles, coated ferromagnetic microparticles and nanoparticles, as well as other magnetic beads known in the art. In another aspect, the probe is an antibody. The antibody may bind to surface markers on viral particles. In particular, the antibody may bind to Spike glycoprotein (S1 & S2) of SARS-Cov-2, GP41 or GP120 of Lentivirus, other known viral particle surface markers, or a combination thereof. In another aspect, the first diameter may be between about 5 nm and about 300 nm, about 5 nm and about 200 nm, about 10 nm and about 300 nm, about 10 nm and about 200 nm, about 10 nm and about 150 nm, about 10 nm and about 100 nm, about 10 nm and about 50 nm, about 20 nm and about 300 nm, about 20 nm and about 200 nm, about 20 nm and about 100 nm, or about 50 nm and about 200 nm. In a particular aspect, the first diameter may be between about 10 nm and about 200 nm. The second diameter may be less than about 5 μm, less than about 4 μm, less than about 3 μm, less than about 2 μm, less than about 1 μm, or less than about 0.5 μm. In a particular aspect, the second diameter may be less than about 2 μm. The nanopores may be arranged in an array with a regular pattern or an irregular arrangement. And some of the baffles may be larger than other ones. In another aspect, the membrane is formed from one or more materials comprising one or more of a polyethylene terephthalate (PET), a polycarbonate (PC), a polypropylene (PP), a polyimides (PI), or a polyethersulphone (PES). In another aspect, the system further comprises a third chamber and a filter positioned between the third chamber and the first chamber, the filter comprising a first filter surface facing and at least partially defining the third chamber, a second filter surface facing and at least partially defining the first chamber and a plurality of filter pores extending between the first and second filter surfaces. In another aspect, each filter pore may have a diameter of 150 nm to 6 microns, 150 nm to 5 microns, 200 nm to 5 microns, 200 nm to 4 microns, 200 nm to 3 microns, 200 nm to 2 microns, 200 nm to 1 micron, 300 nm to 5 microns, 400 nm to 5 microns, 500 nm to 5 microns, 600 nm to 5 microns, 700 nm to 5 microns, 800 nm to 5 microns. 900 nm to 5 microns, or 1000 nm to 5 microns. In another aspect, the filter is formed from one or more materials comprising a polyethylene terephthalate (PET), a polycarbonate (PC), a polypropylene (PP), a polyimides (PI), and a polyethersulphone (PES). In another aspect, the device for inducing fluid flow generates a flow rate of between about 0.01 ml/hour to about 1000 mL/hour, about 0.01 mL/hour to about 900 mL/hour, about 0.01 mL/hour to about 800 mL/hour, about 0.01 ml/hour to about 700 mL/hour, about 0.01 mL/hour to about 600 mL/hour, about 0.01 mL/hour to about 500 mL/hour, about 0.01 mL/hour to about 400 mL/hour, about 0.01 mL/hour to about 300 mL/hour, about 0.01 mL/hour to about 200 mL/hour, about 0.01 mL/hour to about 100 mL/hour, about 0.05 mL/hour to about 1000 mL/hour, about 0.1 mL/hour to about 1000 mL/hour, about 0.2 mL/hour to about 1000 mL/hour, about 0.3 mL/hour to about 1000 mL/hour, about 0.4 mL/hour to about 1000 mL/hour, or about 0.5 mL/hour to about 1000 mL/hour. In another aspect, the device for inducing fluid flow generates a pressure less than about 0.3 atm, less than about 0.4 atm, less than about 0.5 atm, less than about 1 atm, less than about 1.1 atm, less than about 1.2 atm, less than about 1.3 atm, less than about 1.4 atm, less than about 1.5 atm. In particular, the device for inducing fluid flow generates a pressure less than about 1 atm. In another aspect, the device for inducing fluid flow comprises a syringe pump, an electroosmotic pump, a micropump, a centrifuge, or a combination thereof. In another aspect, the sample is applied perpendicularly or tangentially to the membrane or the filter. In another aspect, the viral particles are about 60-200 nm, about 65-200 nm, about 70-200 nm, about 75-200 nm, about 80-200 nm, about 60-150 nm, about 65-150 nm, about 70-150 nm, about 75-150 nm, about 80-150 nm, about 60-100 nm, about 65-100 nm, about 70-100 nm, about 75-100 nm, or about 80-100 nm in size. In another aspect, the system described herein can be used to isolate viral particles that cause viral infectious diseases that include, but are not limited to, Paramyxoviridae (respiratory syncytial virus (RSV), parainfluenza virus (Ply), metapneumovirus (MPV), enteroviruses), Picornaviridae (Rhinovirus, RV), Coronaviridae (CoV), Adenoviridae (Adenovirus), Parvoviridae (HBoV), Orthomyxoviridae (influenza A, B, C, D, Isavirus, Thogotovirus, Quaranjavirus), Herpesviridae (human herpes viruses, Varicella zoster virus, Epstein-Barr virus, cytomegalovirus), avian influenza, smallpox, pandemic influenza, adult respiratory distress syndrome (ARDS). CoV can include one or more of Severe Acute Respiratory Syndrome (BARS-CoV), Middle East Respiratory Syndrome (MERS-CoV), COVID-19 (2019-nCoV, SARS-CoV-2), 229E, NL63, OC43, or HKU1. In another aspect, the viral particles are SARS-COV-2 viral particles.

Another embodiment described herein is a method for isolating viral particles comprising: providing a system as described herein, and inducing fluid flow through the membrane from the first chamber to the second chamber, whereupon the viral particles are isolated in the second chamber.

Another embodiment described herein are viral particles isolated using the methods described herein.

Another embodiment described herein is a method for isolating viral particles comprising: providing a system comprising: a first chamber; a second chamber; a third chamber; a membrane positioned between the first and second chambers, and comprising a first membrane surface facing and at least partially defining the first chamber, a second membrane surface facing and at least partially defining the second chamber and a plurality of asymmetrically shaped nanopores extending between the first and second membrane surfaces, wherein each nanopore includes a first nanopore opening at the first membrane surface having a first diameter, and a second nanopore opening at the second membrane surface having a second diameter that is greater than the first diameter; a filter positioned between the third chamber and the first chamber, the filter comprising a first filter surface facing and at least partially defining the third chamber, a second filter surface facing and at least partially defining the first chamber and a plurality of filter pores extending between the first and second filter surfaces; and a device for inducing fluid flow through the filter from the third chamber to the first chamber and through the membrane from the first chamber to the second chamber by pressure driven flow, electroosmotic flow, centrifugal force, or a combination thereof; introducing a sample comprising viral particles into the third chamber; inducing fluid flow through the filter and the membrane from the third chamber to the first chamber and from the first chamber to the second chamber, whereupon the viral particles pass through the filter and are isolated in the second chamber. In one aspect, the sample comprising viral particles comprises one or more of cell culture supernatants or a sample obtained from an animal subject. In another aspect, the sample obtained from an animal subject comprises one or more of blood, saliva, droplets from coughing, droplets from sneezing, plasma, tear, serum, urine, sputum, pleural effusion, or ascites. In another aspect, the first diameter is between about 10 nm to about 200 nm. In another aspect, the second diameter is less than about 2 μm. In another aspect, the membrane is formed from one or more materials comprising a polyethylene terephthalate (PET), a polycarbonate (PC), a polypropylene (PP), a polyimides (PI), or a polyethersulphone (PES). In another aspect, each filter pore has a diameter of 200 nm to 5 microns. In another aspect, the filter is formed from one or more materials comprising a polyethylene terephthalate (PET), a polycarbonate (PC), a polypropylene (PP), a polyimides (PI), or a polyethersulphone (PES). In another aspect, the first chamber comprises a wall opposite of the first membrane that comprises one or more baffles. In another aspect, the device for flowing the sample generates a flow rate of between about 0.01 mL/hour to about 1000 mL/hour, In another aspect, the device for inducing fluid flow generates a pressure less than about 1 atm. In another aspect, the device for inducing fluid flow comprises a syringe pump, an electroosmotic pump, a micropump, a centrifuge, or a combination thereof.

In another aspect, the sample is applied perpendicularly or tangentially to the filter.

Another embodiment described herein are viral particles isolated using any of the methods described herein.

Another embodiment described herein is a method for detecting viral particles in a sample comprising providing the system as described herein, inducing fluid flow through the membrane from the first chamber to the second chamber, whereupon the viral particles are isolated in the second chamber, lysing the isolated viral particles, and, measuring viral RNA. In another aspect, the isolated viral particles are lysed using chemical or thermal lysing. In another aspect, when chemical lysing is used, RNA extraction is performed on the isolated viral particles before the viral RNA is measured. In another aspect, when thermal lysing is used, the viral RNA is directly measured. In another aspect, the sample has an initial volume of about 1 mL to about 100 mL. In a particular aspect, the sample has an initial volume of about 2.5 mL to about 10 mL. In another aspect, the sample is collected by a swab. In another aspect, the sample is extracted from the swab in a buffer.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All the various embodiments, aspects, and options disclosed herein can be combined in any variations or iterations. The scope of the compositions, formulations, methods, and processes described herein include all actual or potential combinations of embodiments, aspects, options, examples, and preferences herein described. The compositions, formulations, or methods described herein may omit any component or step, substitute any component or step disclosed herein, or include any component or step disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the specification discloses and describes merely exemplary embodiments. All patents and publications cited herein are incorporated by reference herein for the specific teachings thereof.

Various embodiments and aspects of the inventions described herein are summarized by the following clauses:
Clause 1. A system for isolating viral particles comprising: a first chamber; a second chamber; a membrane positioned between the first and second chambers, and comprising a first membrane surface facing and at least partially defining the first chamber, a second membrane surface facing and at least partially defining the second chamber and a plurality of asymmetrically shaped nanopores extending between the first and second membrane surfaces, wherein each nanopore includes a first nanopore opening at the first membrane surface having a first diameter, and a second nanopore opening at the second membrane surface having a second diameter that is greater than the first diameter; a sample comprising the viral particles positioned within the first chamber; and a device for inducing fluid flow through the membrane from the first chamber to the second chamber by pressure driven flow, electroosmotic flow, centrifugal force, or a combination thereof.
Clause 2. The system of clause 1, wherein the first membrane surface comprises one or more baffles.
Clause 3. A system for isolating viral particles comprising: a first chamber; a second chamber; a membrane positioned between the first and second chambers, and comprising a first membrane surface facing and at least partially defining the first chamber, a second membrane surface facing and at least partially defining the second chamber and a plurality of asymmetrically shaped nanopores extending between the first and second membrane surfaces, wherein each nanopore includes a first nanopore opening at the first membrane surface having a first diameter, and a second nanopore opening at the second membrane surface having a second diameter that is greater than the first diameter; wherein the first membrane surface comprises one or more baffles; a sample comprising the viral particles positioned within the first chamber; and a device for inducing fluid flow through the membrane from the first chamber to the second chamber by pressure driven flow, electroosmotic flow, centrifugal force, or a combination thereof.
Clause 4. The system of any one of clauses 1-3, wherein the first membrane surface is coated with a magnetic alloy.
Clause 5. The system of any one of clauses 1-4, wherein the first diameter is from about 10 nm to about 200 nm.
Clause 6. The system of any one of clauses 1-5, wherein the first diameter of the plurality of asymmetrically shaped nanopores has a coefficient of variation of less than 10% between each nanopore.
Clause 7. The system of any one of clauses 1-6, wherein the second diameter is from about 30 nm to about 10 μm.
Clause 8. The system of any one of clauses 1-7, wherein a distance between the first and second membrane surfaces is from about 1 μm to about 100 μm.
Clause 9. The system of any one of clauses 1-8, wherein the membrane comprises a nanopore density from about 10⁶ to about 10¹⁰ nanopores/cm².
Clause 10. The system of any one of clauses 1-9, wherein the nanopores of the membrane are ion-etched.
Clause 11. The system of any one of clauses 1-10, wherein the first chamber comprises a plurality of inlets.
Clause 12. The system of any one of clauses 1-11, wherein the first chamber comprises a first inlet for loading of the sample into the first chamber; and, a second inlet for loading of an elution buffer, lysing solution, PCR cocktail, or a combination thereof into the first chamber; and, wherein a concentrated virus solution is eluted from the first chamber through the first inlet or the second inlet into a collection tube or a third chamber.
Clause 13. The system of clause 12, wherein the first inlet and second inlet are the same inlet.
Clause 14. The system of any one of clauses 1-13, wherein the second chamber comprises an outlet wherein the device for inducing fluid flow through the membrane from the first chamber to the second chamber is connected.
Clause 15. The system of any one of clauses 1-14, wherein the membrane is formed from one or more materials comprising one or more of a polyethylene terephthalate (PET), a polycarbonate (PC), a polypropylene (PP), a polyimides (PI), or a polyethersulphone (PES).
Clause 16. The system of any one of clauses 1-15, further comprising a fourth chamber and a filter positioned between the fourth chamber and the first chamber, the filter comprising a first filter surface facing and at least partially defining the fourth chamber, a second filter surface facing and at least partially defining the first chamber and a plurality of filter pores extending between the first and second filter surfaces,
Clause 17. The system of clause 16, wherein each filter pore has a diameter of about 200 nm to about 5 microns.
Clause 18. The system of clause 16 or clause 17, wherein the filter is formed from one or more materials comprising a polyethylene terephthalate (PET), a polycarbonate (PC), a polypropylene (PP), a polyimides (PI), and a polyethersulphone (PES).
Clause 19. The system of any one of clauses 1-18, wherein the device for inducing fluid flow generates a flow rate of about 0.01 mL/hour to about 100 mL/hour.
Clause 20. The system of any one of clauses 1-19, wherein the device for inducing fluid flow generates a pressure less than about 1 atm.
Clause 21. The system of any one of clauses 1-20, wherein the device for inducing fluid flow comprises a syringe pump, an electroosmotic pump, a micropump, a centrifuge, a vacutainer, a snap lock syringe pump, or a combination thereof.
Clause 22. The system of any one of clauses 1-21 wherein the sample is applied perpendicularly or tangentially to the membrane or the filter.
Clause 23. The system of clause 22, wherein when the sample is applied tangentially to the membrane or filter, a flow rate of about 5 mL/hour to about 40 mL/hour.
Clause 24. The system of any one of clauses 1-23, wherein the viral particles are about 80-100 nm in size.
Clause 25. The system of any one of clauses 1-24, wherein the viral particles are SANS-COV-2 viral particles.
Clause 26. The system of any one of clauses 4-25, wherein the magnetic alloy is nickel-iron, samarium-cobalt, aluminum-nickel-cobalt, nickel-iron-chromium, iron-chromium-cobalt, or neodymium-iron-boron.
Clause 27. The system of any one of clauses 4-26, wherein the viral particles are bound to a probe that is coupled to a magnetic bead.
Clause 28. The system of clause 27, wherein the probe is an antibody.
Clause 29. The system of any one of clauses 1-28, wherein the system is connected with a plurality of identical systems in series or in parallel.
Clause 30. A use of the system of any one of clauses 4-29, for isolating a virus.
Clause 31. A method for isolating viral particles comprising: providing the system of any of clauses 1-30, and inducing fluid flow through the membrane from the first chamber to the second chamber, whereupon the viral particles are isolated in the second chamber.
Clause 32. A viral particle isolated using the method of clause 31.
Clause 33. A method for detecting viral particles in a sample comprising: providing the system of any of clauses 1-32; inducing fluid flow through the membrane from the first chamber to the second chamber, whereupon the viral particles are isolated in the second chamber; lysing the isolated viral particles; and measuring viral RNA.
Clause 34. The method of clause 33, wherein the isolated viral particles are lysed using chemical, mechanical, or thermal lysing.
Clause 35. The method of clause 34, wherein when chemical lysing is used, RNA extraction is performed on the isolated viral particles before the viral RNA is measured.
Clause 36. The method of clause 34, wherein when thermal or mechanical lysing is used, the viral RNA is directly measured.
Clause 37. The method of clause 34, wherein the lysed viral particles are mixed with a PCR cocktail in the first chamber.
Clause 38. The method of any one of clauses 33-37, wherein the sample has an initial volume of about 1 to about 100 mL.
Clause 39. The method of any one of clauses 33-38, wherein the sample is collected by a swab.
Clause 40. The method of clause 39, wherein the sample is extracted from the swab in a buffer.
Clause 41. The method of any one of clauses 33-40, wherein the sample comprising the viral particles comprises one or more of cell culture supernatants or a sample obtained from an animal subject.
Clause 42. The method of clause 41, wherein the sample obtained from an animal subject comprises one or more of blood, saliva, droplets from coughing, droplets from sneezing, plasma, tear, serum, urine, sputum, pleural effusion, or ascites,

### REFERENCES

1. Kucirka et al., “Variation in False-Negative Rate of Reverse Transcriptase Polymerase Chain Reaction-Based SARS-CoV-2 Tests by Time Since Exposure,” Annals of Internal Medicine (2020).
2. Furukawa et al., “Evidence supporting transmission of severe acute respiratory syndrome coronavirus 2 while presymptomatic or asymptomatic,” Emerg Infect Dis. (2020).
3. CDC 2019-Novel Coronavirus (2019-nCoV) Real-Time RT-PCR Diagnostic Panel.
4. Esbin et al, “Overcoming the bottleneck to widespread testing: A rapid review of nucleic acid testing approaches for COVID-19 detection,” RNA (2020).
5. Wölfel et al., “Virological assessment of hospitalized patients with COVID-2019,” Nature (2020).
6. Merindol et al., “SARS-CoV-2 detection by direct rRT-PCR without RNA extraction,” Journal of Clinical Virology 128: 104423 (2020).
7. Smyrlaki et al. “Massive and rapid COVID-19 testing is feasible by extraction-free SARS-CoV-2 RT-PCR,” Nature Comm. 11: 4812 (2020).

### EXAMPLES

### Example 1

### General Methods

### Asymmetric Nanopore Membrane (ANM)

Polycarbonate (PC) track-etched membranes were prepared by the track-etching technique, which is based on the irradiation of a material with swift heavy ions and subsequent chemical etching. The pore size can be controlled by the etching time, and the number of ions per unit area determines the number of damage tracks and, hence, pores. Polycarbonate membranes of this type having cylindrical pores with diameters ranging from as small as 10 nm to as large as 20 μm, and pore densities as high as 5×10³ cm⁻², are sold commercially. 30±3 nm PC membranes were used in this study and were 6-μm-thick and obtained from Sigma (Whatman Nuclepore Track-Etched Membranes; WHA110602). The as-received membranes have a cylindrical pore shape and have a pore density of 5×10⁸ cm⁻². The pore size and density of the as-received membranes have been confirmed by SEM. Asymmetric nanopores were produced by a simple O₂ plasma etching process on one face of the as-received tracked membrane. The asymmetric etching forms a cone-like asymmetric pore shape. A 25 mm-in-diameter cylindrical pore membrane was placed on a silicon wafer (500 μm thick). One surface of these membranes appears shiny and the opposite surface appears rough to the eye. The membrane was placed on the silicon wafer with the rough surface up. A 2.5 cm×2.5 cm PMMA sheet that had a 21 mm-in-diameter hole cut through it was placed on top of the membrane, and Kapton tape was used to attach the PMMA sheet to the silicon wafer. This hole defined the area of the membrane exposed to the O₂ plasma. O₂ plasma etching was performed with a commercial reactive ion etch system (Oxford PlasmaPro System, model RIE100 or Plasmatherm 790 RIE). The etching conditions were as follows: O₂ gas pressure 200 Pa, gas flow rate 30 standard cm³ min⁻¹, and power 100 W. Plasma etching enlarges the pore diameter at the upper surface (base side) at a high etching rate of 50 nm/min while the etching of the pore diameter at the lower surface only occurs after the plasma penetrates the membrane at a much lower etching rate ˜5 nm/min, but the pore diameter remains unchanged at the lower surface. Furthermore, plasma etching also reduces the thickness of the membrane. 25 mm diameter ANMs with an average pore diameter of 60 nm were used for high-yield virus isolation. The SARS-CoV-2 have an average size of 100 nm. As a result, the ANMs for virus isolation must have a pore size of less than 100 nm. It was found that ANMs with an average pore size of 60 provides the highest virus recovery rate.

### Lentivirus Isolation

Lentivirus stocks (Takara Bio USA, Inc. #0038VCT) were first diluted 100 times with 1×PBS. Before experiments, 1 μL of the diluted lentivirus solution was added into 1 mL. 1×PBS for the working solution. 1 μL of the working solution was spiked into the test samples (PBS, Viral Transport Medium, saliva, plasma). The purchased virus stocks had a 1×10⁹-1×10¹⁰ TU/mL of lentivirus. 10-100 TU of lentiviruses were used in the final spiked samples. (TU: Transducing Units).

Virus isolation was performed by direct flow nanofiltration using the as-prepared asymmetric nanopore membranes. The membrane was sealed in a home-made plastic membrane holder. The plastic housing was secured with metal screws and nuts, and a plastic ring-shaped gasket provided a leak-free seal. The isolation involved size-based isolation and washing steps. 25 mm-in-diameter ANMs with an average pore diameter of 60 nm were used for high-yield lentivirus isolation. The lentiviruses used in this application have an average size of 100 nm. As a result, the ANMs for lentivirus isolation must have a pore size smaller than 100 nm. ANMs with smaller size are expected to offer better retention performance for virus isolation. But ANMs with smaller size have several disadvantages including low throughput due to high hydrodynamic resistance and increased virus damage/loss due to higher pressure drop at the pore tip of ANM which can lyse the viruses, Thus, there is an optimized pore size of ANM used for certain virus isolation. In this application, ANMs with an average pore size of 60 was found to offer the highest virus recovery rate. The virus samples were introduced continuously into the asymmetric nanopore membrane filtration device via a syringe using a syringe pump at a constant flow rate (60 mL/h), followed by a 5 mL 1×PBS washing step. The concentrated viruses were recovered from the fluid chamber next to the asymmetric nanopore membrane, and the isolated viruses were then used for downstream PCR analysis.

Upstream filters, tangential-flow ANM filtration with baffle design, and/or magnetic beads will be necessary when isolating viruses from highly heterogeneous samples such as serum, plasma. The virus samples may be prefiltered with a PES syringe filter. Virus isolation can also be performed in a tangential-flow nanofiltration mode when large-volume and heterogeneous samples are processed. Filter-cake formation and high build-up pressure lead to virus lysing and coalescence especially when the highly heterogeneous samples are filtered in large volume/ In the tangential-flow nanofiltration assay, the feed stream passes parallel to the asymmetric nanopore membrane face as one portion passes through the membrane (permeate) while the remainder (retentate) is recirculated. A peristaltic pump recirculates the retentate stream at a constant flow rate to prevent the formation of a restrictive layer, followed by a wash step comprising up to 30 mL 1×PBS. The ANM flow chip was made by 3D printing the chip with a channel dimension of 65 (L)×20 (W)×1 (H) mm, A baffled tangential flow design was also introduced to better suppress the fouling and filter cake formation. These baffles were fabricated on the top wall of the flow channel such that the baffles are part of the flow chamber which is made of polymethyl methacrylate (PMMA). The baffles can be shaped like cubes or triangular prisms. The baffles can have a height ranging from about 25 μm to about 2 mm and be spaced from about 100 μm to about 5 mm apart. The baffle design allowed for a different shear rate and polarized layer length of the filter cake at the baffle and the spacing between the baffle. The difference in characteristic polarized length and shear rate of the filter cake allows it to break at the point of change, A two-dimensional baffle can also induce vortices in the system that breakup the filter cake. The baffle design was inspired by a specialized filtering structure in filter feeder (e.g., suspension feeding) fish, the specialized filtering structure can significantly enhance the restrictive clogging layer removal by inducing localized vortices.

### SARS-CoV-2 Viruses and Reagents

Heat-inactivated SARS-CoV-2 were obtained from BEI Resources (NR-52286, Lot: 70037779). The purchased virus stock has a concentration of 1.77×10⁸ genome equivalents/mL quantified using BioRad QX200 Droplet Digital PCR (ddPCR™) System. Before experiments, the virus stock was diluted by a factor ranging from 1000 to 10,000,000 to obtain virus solution with various spiked virus concentrations. The viral transport medium was made with 0,5% bovine serum albumin (BSA), benzylpenicillin (2×10⁶ IU/L), streptomycin (200 mg/L), polymyxin B (2×10⁶ IU/L), gentamicin (250 mg/L), nystatin (0.5×10⁶ IU/L), ofloxacin hydrochloride (60 mg/L), and sulfamethoxazole (0.2 g/L) in the Hank's Balanced Salt Solution (HBSS). Triton X-100 in the HBSS was used in the viral RNA extraction.

### Concentrating SARS-CoV-2 and Extracting Viral RNA

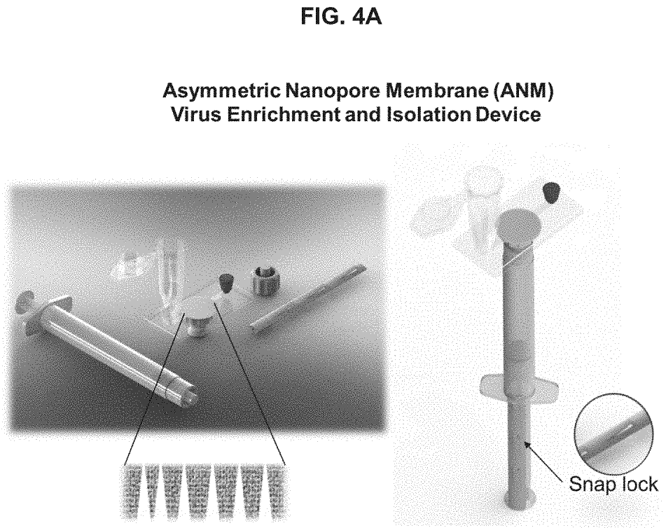

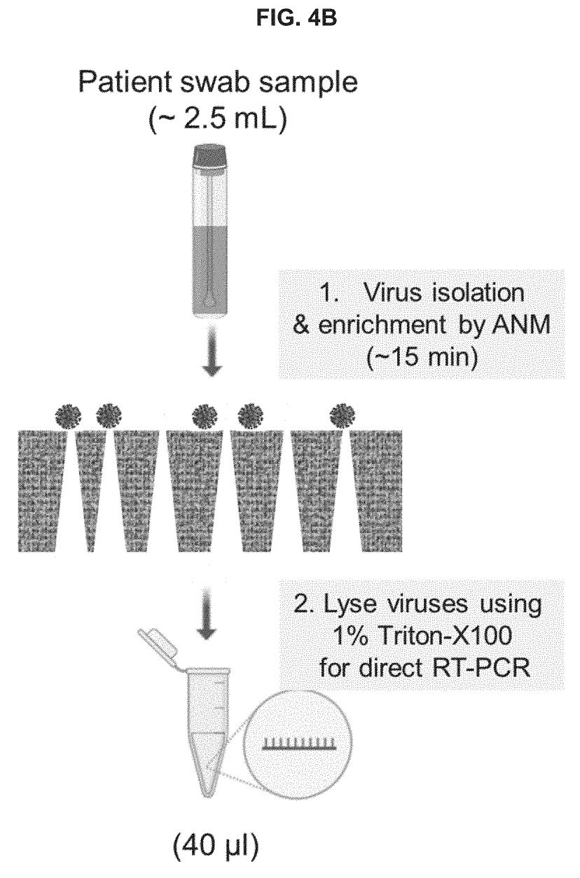

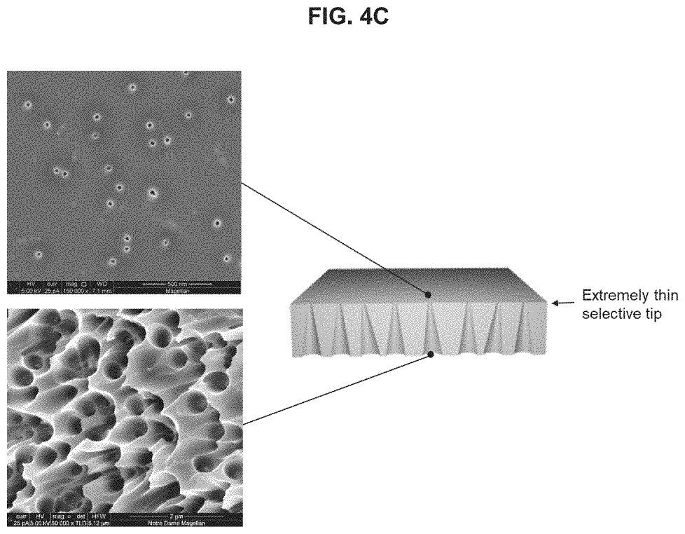

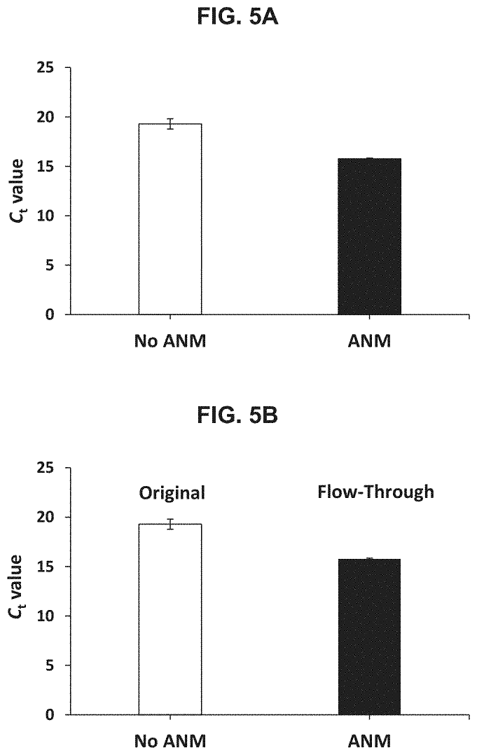

The ANM virus enrichment and isolation device is composed of two components: an ANM holder and a syringe with a snap lock design as shown in FIG. 4A-B. The ANM holder was fabricated by 3D printing in which the ANM separates the device into a top chamber and a bottom chamber. The ANM device has two inlets for sample and elution buffer loading, respectively, for the top chamber and an outlet for permeate flow for the bottom chamber, A syringe is connected to the outlet of the bottom chamber and the syringe with a snap lock was used to provide a negative pressure to transport the virus sample solution through the ANM for concentrating and purifying the virus, Snap Lock helps eliminate having to hold the plunger during aspiration, In a typical virus concentration experiment, a syringe loaded with 2.5 mL virus sample was connected with the sample loading inlet. All the air in the top chamber was then driven out by manually pushing the plunger of the sample syringe and the elution buffer loading inlet was sealed with a cap once the top chamber was filled with sample solution. To initiate the concentration process, the snaps lock was inserted into the plunger assembly and the plunger was pulled until it snapped locked to provide pressure to pump all of the virus sample solution through the ANM while concentrating and purifying the virus. Finally, the concentrated viruses were eluted by pipetting a small volume (˜40 μL) of buffer into the top chamber through the elution buffer loading inlet. For direct RT-PCR, 1% Triton X-100 was used to lyse the viral particles and elute the viral RNA.

### Magnetic Asymmetic Nanopore Membrane (MNM) Virus Isolation

Briefly, 80 nm Au was deposited using a thermal evaporator (Oerlikon Leybold 8-pocket electron-beam) onto one side of a 450 nm track-etched polycarbonate membrane (Whatman) to provide a working electrode in the subsequent electrodeposition process. Then 200 nm Ni₈₀Fe₂₀ film was electrodeposited on top of the Au film. An Ni electrode was used in the electrodeposition solution. Ni₈₀Fe₂₀ electrodeposition solution was composed of 289 g/L NiSO₄.6H₂O, 64 g/L FeSO₄.7H₂O, 40 g/L H₃BO₃, 8.9 g/L 5-sulfosalicylic acid dihydrate, and 3 g/L 1,3,(6,7)-naphthalenetrisulfonic acid trisodium salt hydrate. During the electrodeposition, the deposition current <2.5 mA/cm². The resulting MNM has an asymmetric geometry with a base diameter of about 450 nm and a tip diameter of about 250 nm.

### Virus Isolation Using MNM

Viruses will first be isolated based on their size using ANM, as detailed herein. Immunosorting of viruses will be performed by positive selection using magnetic nanobeads recognizing proteins specific to the virus. These magnetic nanobeads (20-30 nm) with antibodies will be added to the sample (isolated viruses) and incubated for 30 min at room temperature with shaking. Then the samples will be added to the reservoir of the MNM holder and pressure will be applied by a programmable syringe pump to pump the virus sample at a flow rate of 1 mL/h. The MNM holder was fabricated by a computer-controlled milling machine (Roland, monoFab SRM-20). Two ring neodymium magnets were placed on the top and bottom side of the MNM holder, respectively, which provide the magnetic field to magnetize the MNM. As the sample solution will be pumped through the chip, viruses that are labeled with magnetic nanoparticles will be captured at the edge of the pores of the MNM.

### RNA Extraction

RNA was isolated from samples using the NucleoSpin® RNA Virus Kit (Takara Blo) according to the manufacturer's manual. 50 μL of a sample was first mixed with 200 μL RAV1 solution and incubated at 70° C. for 5 min. After adding 200 μL of ethanol, the solution was transferred into the binding column and centrifuged at 8,000×g for 1 min. The column was then washed with 500 μL RAW and 600 μL RAV3 sequentially at 8,000×g for 1 min, followed by 200 μL RAV3 washing and drying at 11,000×g for 5 min. Finally, 50 μL of at 70° C. RNase-free water was added to elute the RNA at 11,000×g for 1 min after incubation at room temperature for 2 min.

qRT-PCR

The lysed virus samples were collected from the device as described herein and were analyzed by one-step qRT-PCR. The experiments were carried out using Lenti-X™ qRT-PCR Titration Kit (Takara Bio USA) on a StepOnePlus™ Real-Time PCR System (Applied Biosystems) was used for quantification of lentivirus according to the manufacturer's manual. The Lenti-X™ qRT-PCR Titration Kit manufacturer does not disclose the primer sequences and therefore, the sequence information is unavailable. Each reaction contained 2 μL collected sample, 8 μL RNase-Free Water, 12.5 μL Quant-X Buffer (Takara Bio USA), 0.5 μL Lenti-X Forward Primer (Takara Bio USA, 10 μM), 0.5 μL Lenti-X Reverse Primer (Takara Bio USA, 10 μM), 0.5 μL ROX Reference Dye LMP (50×) (Takara Bio USA), 0.5 μL Quant-X™ Enzyme (Takara Bio USA), and 0.5 μL RT Enzyme Mix (Takara Bio USA) in a final volume of 25 μL. The reaction mixtures were incubated for 5 min at 42° C. for reverse transcription, quenched at 95° C. for 10 s, followed by 40 qPCR cycles at 95° C. for 5 s and 60° C. for 30 s. The C_{q} values were acquired and analyzed using StepOne™ Software v2.3 in accordance with the MIQE guidelines.

TaqPath 1-step RT-qPCR master mix (ThermoFisher, A15299) and 2019-nCoV RUO Kit (IDT) were used for quantification of SARS-CoV-2 according to the manufacturers manuals, Each reaction contained 2 μL sample, 5 μL TaqPath 1-step RT-qPCR master mix, 11.5 μL RNase-free water, and 1.5 μL N1/N2 probes in a final volume of 20 μL. The N1/N2 primer sequences are shown in Table 1. The reaction mixtures were incubated at 25° C. for 2 min, 50° C. for 15 min, and 95° C. for 2 min followed by 45 cycles of 95° C. for 3 sec, and 55° C. for 30 sec on a StepOnePlus™ Real-Time PCR System (Applied Biosystems). For absolute quantification, standard curves were generated from a series of dilutions of standard RNA Control (AcroMetrix Coronavirus 2019 (COVID-19) RNA Control (RUO), Thermo, 954519) for each plate. The C_{q} values were acquired and analyzed using StepOne™ Software v2.3 in accordance with the MIQE guidelines.

TABLE 1
SARS-CoV-2 Primers

| Name | Sequence (5′-3′) | SEQ ID NO |
|---|---|---|
| N1 Fwd | GACCCCAAAATCAGCGAAAT | SEQ ID NO: 1 |
| N1 Rev | TCTGGTTACTGCCAGTTGAATCTG | SEQ ID NO: 2 |
| N1 Probe | FAM-ACCCCGCAT/ZEN/TACGTTTGGTGGACC-3IABkFQ | SEQ ID NO: 3 |
| N2 Fwd | TTACAAACATTGGCCGCAAA | SEQ ID NO: 4 |
| N2 Rev | GCGCGACATCCGAAGAA | SEQ ID NO: 5 |
| N2 Probe | FAM-ACAATTTGC/ZEN/CCCCAGCGCTTCAG-3IABkFQ | SEQ ID NO: 6 |
| Target | SARS-CoV-2/human/USA/WA-CDC-WA1/2020, | SEQ ID NO: 7 |
| | complete genome GenBank: MN985325.1 | |

FAM: 5′ 6-FAM (fluorescein dye); /ZEN/: ZEN fluorescent quencher located between N9 and N10; 3IABkFQ: 3′-Iowa Black ® Fluorescence Quencher

### Example 2

### Virus Concentration Using the ANM

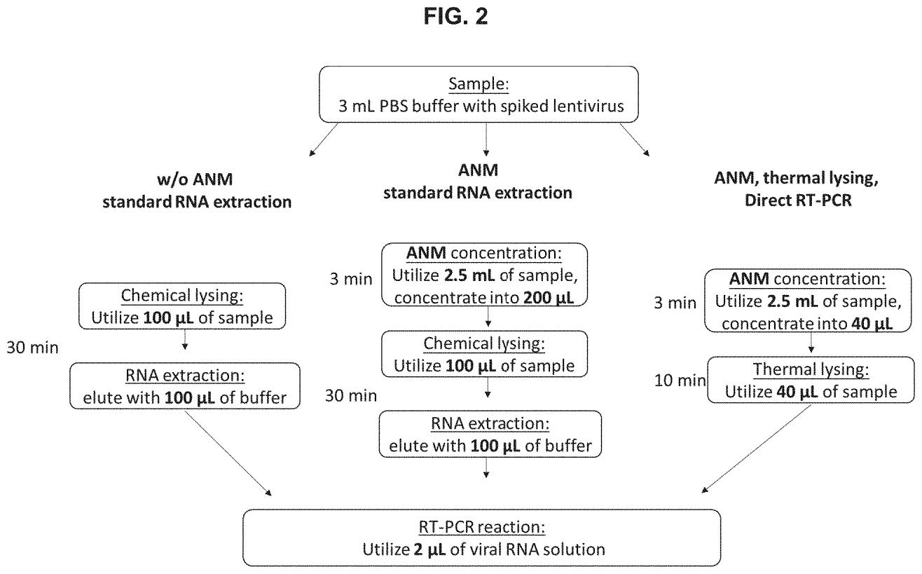

The standard RT-PCR method (standard RNA extraction) with or without ANM concentration was compared. Furthermore, the possibility of the direct RT-PCR method (ANM concentration and thermal lysing), as illustrated in FIG. 2, was assessed. 3 mL of PBS buffer spiked with lentiviruses was used to mimic the VTM from a swab sample. If the lentivirus in the 3 mL PBS buffer can be enriched into a smaller volume (e.g., 100 μL) with high yield, more viral particles can be collected for downstream RNA extraction and RT-PCR analysis, improving the sensitivity by a factor proportional to the enrichment factor. Moreover, the size-based ANM filtration only concentrates and isolates the viral particle and removes the interfering reagents which are most likely in smaller size, opening up the possibility that RNA extraction can be bypassed and the concentrated viral particles can simply be thermally lysed and then used for direct RT-PCR. Eliminating the RNA extraction step allows a reduction in both the cost and time for sample preparation. The current RNA extraction step used for viral RNA extraction usually takes 30 mins to complete and requires extensive human labor and materials such as RNA extraction kits that are already in short supply.

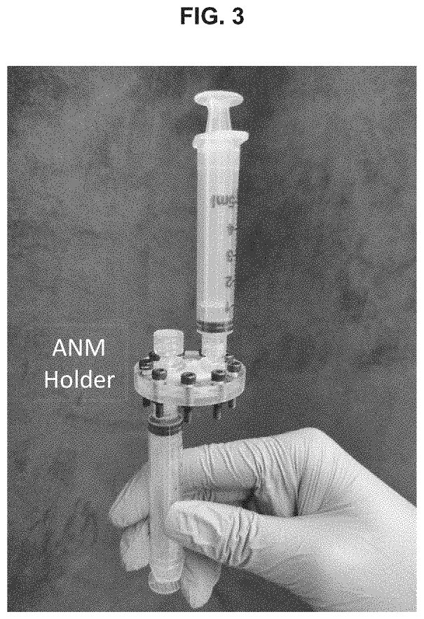

It is worth noting that using PBS buffer as the swab viral particle release medium is advantageous. Unlike the heterogenous VTM that contains serum or BSA, the PBS buffer with viral particles can be driven through the ANM in high throughput and in a filter-cake-free manner, which is the key for high yield concentration. The ANM device disclosed herein (FIG. 3) can be simply driven by a negative pressure, for example, by pulling the bottom syringe. This allows for enrichment of about 2.5 mL of a sample within about 3 minutes. In practice in the real-world, the viral particles on the swab can first be extracted in PBS buffer by vigorously swirling the swab in the buffer. Next, the ANM device can be connected to a vacuum tube (like a blood collection tube) to initiate the concentration process. After the viral particles are trapped on the ANM, about 100 μL of VTM is introduced to elute the viral particles on the membrane. All these steps can be done right after the swab sample is collected from the patient. Here, eluting the viral particles in the VTM is essential for maximizing the amount of virus before it is transported to a test facility. A recent study has found that some VTMs are compatible with direct RT-PCR [6]. All experiments were performed with PBS buffer only, however VTM samples will be tested. RNA extraction and/or RT-PCR was performed right after the ANM sample concentration.

### Example 3

### Concentration of Viruses Using ANM Increases the Sensitivity of Standard RT-PCR Methods

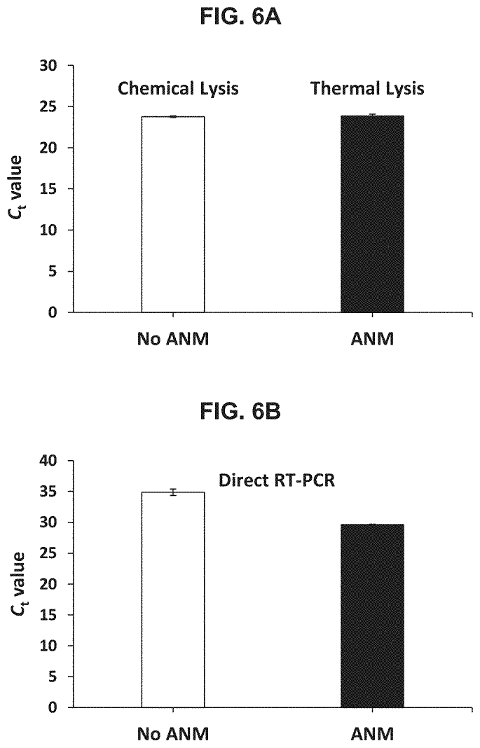

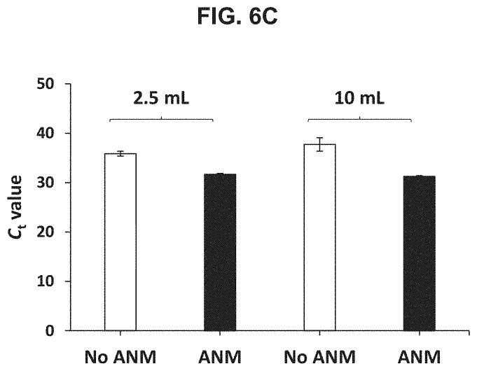

The standard RT-PCR method with or without ANM concentration was compared. The test sample was obtained by spiking lentiviruses into 3 mL PBS buffer (FIG. 2). Then the sample was divided into two groups: 1) 100 μL of sample without ANM concentration and 2) 2.5 mL sample which was then concentrated into 200 μL using ANM (the expected enrichment factor was about 12.5). Next, standard RNA extraction (with chemical lysing) was performed on both sample groups. There was no further concentration in the RNA extraction step since the input volume and elute volume were the same (100 μL). When the sample was concentrated using ANM, Ct was 15.8 compared to a mean of 19.3 when the viruses were in the dilute solution without ANM concentration, indicating a significantly improved sensitivity of RT-PCR using ANM by a factor of 11 (FIG. 6A). Given the 11-fold sensitivity improvement (concentration increase) and the volume enrichment factor of 12.5, the yield of ANM concentration was estimated to be about 88%. The high yield was also confirmed by the RT-PCR experiments on the flow-through from ANM filtration (FIG. 6B). The virus concentration decreased significantly after passing through the ANM (a Ct decrease of 8.6).

### Example 4

### ANM Concentration Allows Direct RT-PCR

The direct RT-PCR method (thermal lysing) was compared with the standard RT-PCR method (chemical lysing). The chemical lysing and thermal lysing using two identical samples provide similar efficiency (FIG. 6A). This observation is consistent with a recent study [6] showing that the RNA extraction step can be eliminated if the sample is stored in a certain buffer and VTM. As such, it is possible to perform direct RT-PCR after viral particle isolation and concentration using ANM. Ideally, all viral particles can be collected from a swab for direct RT-PCR if the sample can be concentrated into a final volume of 5 μL. Practically, handling such a small volume sample in the ANM chip may be challenging. However, a final volume of 40 μL is more practical and easier to handle. When the sample was concentrated from 2.5 mL to 40 μL, the Ct value decreased from 34.9 to 29.6 (a 5.26 decrease), corresponding to a 38-fold sensitivity increase (FIG. 6B). This significant improvement in sensitivity was obtained using a sample with a very low virus concentration (Ct about 34.9, negative control: 37.5). The concentration performance using different input sample volumes (2.5 mL and 10 mL) was also tested. The same amount of lentivirus was spiked into a PBS solution with an initial volume of 2.5 mL and 10 mL, respectively. After the ANM concentration, the same number of viral particles were recovered (FIG. 6C). The ability to process large volume samples allows for pooled screening without diluting the sample and sacrificing the sensitivity. The ANM concentration device disclosed herein will be tested in a real-world setting (with VTM and swabs, etc,). The standard curves over a large virus concentration range will be obtained to verify the sensitivity improvement enabled by ANM in the real-world setting.

### Example 5

### ANM Device and Workflow

The ANM virus enrichment and isolation device is composed of two components: an ANM holder and a syringe with a snap lock design as shown in FIG. 4A-B. The proposed procedure for viral RNA extraction using the ANM device involves: (i) concentration and isolation of viral particles such as SARS-CoV-2 on the surface of ANM; (ii) lysing of the captured viral particles using 1% Triton X-100 and elution of released viral RNA for direct RT-PCR. This disposable virus isolation and enrichment device improves the sensitivity of the current COVID-19 RT-PCR tests while circumventing RNA extraction in the testing procedure via application of the ANM filtration technology. The ANM device as described herein allows for the following: simultaneous virus particle enrichment, contaminant removal, and viral RNA release on a single device; compatibility with the workflow of current FDA-approved RT-PCR tests; and, significant improvements in virus and viral RNA recovery over current clinical processes using a rapid (<15 min), low cost, and disposable device to reduce the rate of false negative test results.

### Example 6

### ANM Allows for Faster Virus Enrichment and Purification

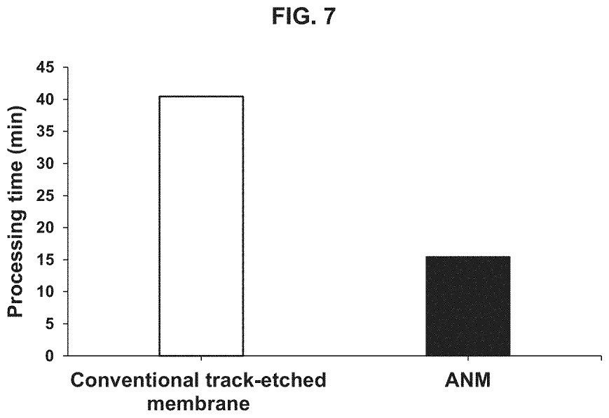

The highly asymmetric nanopore geometry design in the ANM as demonstrated in FIG. 4A-C dramatically reduces hydraulic resistance. Therefore, faster filtration can be achieved with a very low negative pressure (˜0.8 atm), such as that of a vacuum tube produced by a syringe with a snap lock design as shown in FIG. 4A-B. To process 2.5 mL of a viral transport medium (VTM) sample, the filtration time is around 15 min for ANMs, while 40 min is needed for the conventional track-etched nanopore membranes with the same pore size (FIG. 7). Notably, the low pressure also minimizes shear-induced lysis of viral particles, leading to higher RNA recovery. The ANM enables the design of a simple and electronic-component-free device to meet the high-throughput requirements of sample processing.

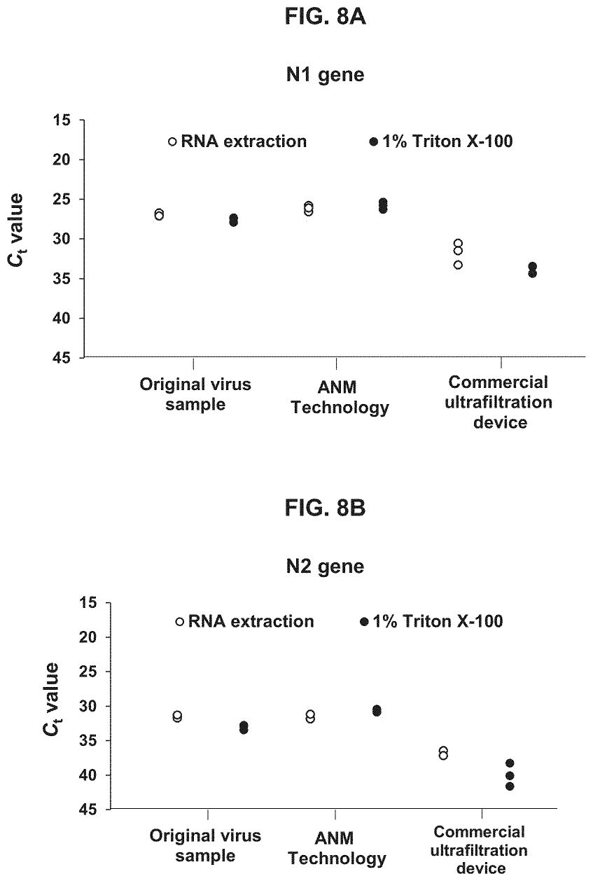

ANMs outperform the conventional ultrafiltration devices. At its core, the ANM contains thin and low-tortuosity (straight) nanopores with a highly asymmetric (conical) geometry and uniform pore tip size as shown in FIG. 4C. As a result, retention is accomplished exclusively by the nanopore orifice with no penetration of virus into the membrane matrix, thus significantly minimizing virus loss in the membrane. The high recovery of the isolated viruses can be simply achieved by retrieving the retentate volume. The ANM technology has vast advantages over conventional ultrafiltration methods; conventional filtration membranes do not allow for high recovery of virus due to viral particle absorption by the membranes as well as loss in the non-uniform and columnar pores. Thus, conventional ultrafiltration membranes have a much lower recovery rate than the ANM as shown in FIG. 8A-B. The ANM can successfully concentrate and recover SARS-CoV-2 viruses (reduced Cₜ value compared to that of the original sample) while significant virus loss was observed (significantly increased Cₜ value) using commercial ultrafiltration devices (Amicon Ultra-2 Centrifugal Filter Unit from Millipore, UFC210024).

### Example 7

### Feasibility of Direct RT-PCR on Surfactant Lysed SARS-CoV-2 Samples

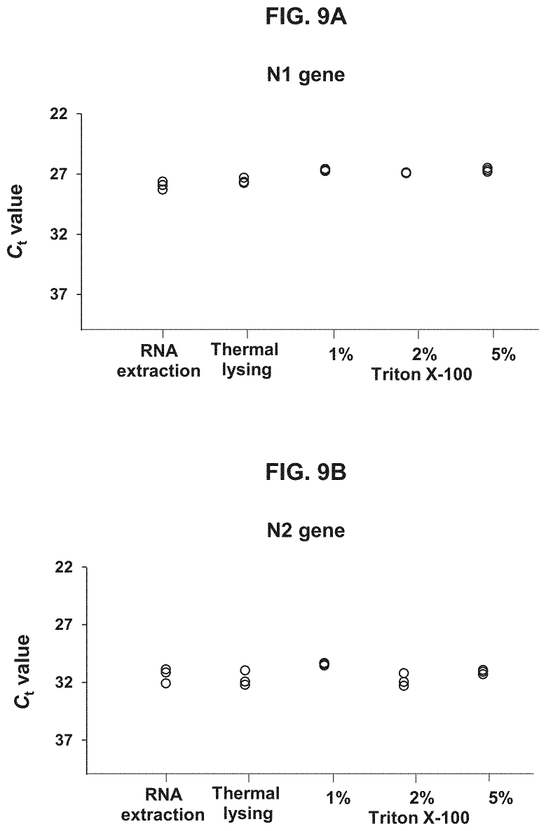

FIG. 9A-B compares direct RT-PCR methods (thermal lysing and surfactant-based lysing using different percentages of Triton X-100) with a standard RNA extraction-based RT-PCR method (chemical lysing). The standard RNA extraction and thermal lysing using two identical samples provide similar efficiency. This observation is consistent with a recent study showing that the RNA extraction step can be eliminated if the sample is stored in a certain buffer and viral transport medium [7]. SARS-CoV-2 are self-assembled particles in which the lipid bilayer is a weak spot. Therefore, the viral envelope can be ruptured by surfactants, As shown in FIG. 9A-B, the lysing performance of Triton X-100 is comparable with the RNA extraction kit and thermal lysing. As such, it is possible to perform direct RT-PCR after viral particle isolation and concentration using the ANM. In the ANM workflow, surfactant-based lysing is preferred to thermal lysing because of its simplicity.

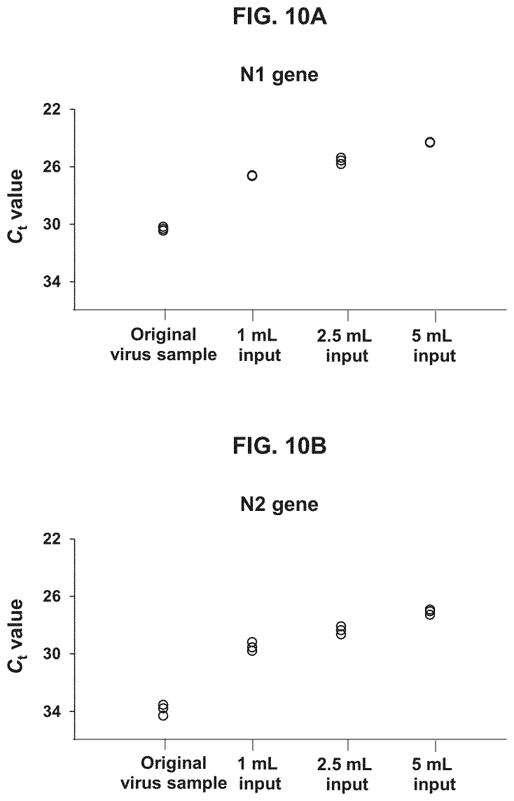

ANMs can process large volume samples and thus boost the assay sensitivity. Ideally, all viral particles from swab samples can be enriched and isolated for direct RT-PCR if the swab sample can be concentrated into a final volume of 5 μL, which is manageable for RT-PCR reactions. Practically, handling such a small volume sample in the ANM device is challenging. A final elution volume of 40 μL is more practical and easier to handle. The enrichment performance of the ANM device was tested using relatively large input sample volumes (1 mL, 2.5 mL, and 5 mL). The Cₜ value for the original virus sample was ˜30.3 for the SARS-CoV-2 nucleocapsid 1 gene (N1 gene). After enrichment with the ANM, the concentration of the final eluted virus samples indeed increased with the input volumes, as indicated by the decreased Cₜ values. As shown in FIG. 10A-B, when the swab samples are concentrated from 1 mL, 2.5 mL, and 5 mL to a final elution volume of 40 μL, the Cₜ values for the Ni gene decreased from 30.3 to 26.6, 25.6, and 24.3, respectively, corresponding to a 13-fold to 64-fold sensitivity increase. Similar results were also shown for the SARS-CoV-2 nucleocapsid 2 gene (N2 gene). It is worth mentioning that such improvement in sensitivity was obtained using a sample with a relatively low concentration of viruses (Cₜ ˜30.3, negative control: 39.5). The ability to enrich a large volume virus sample allows for improvement in the sensitivity of current COVID-19 testing and enables pooled screening without diluting the sample and sacrificing the sensitivity.

### Example 8

ANMs Can Isolate and Concentrate Virus Particles Even in Samples with a Very Low Viral Titer

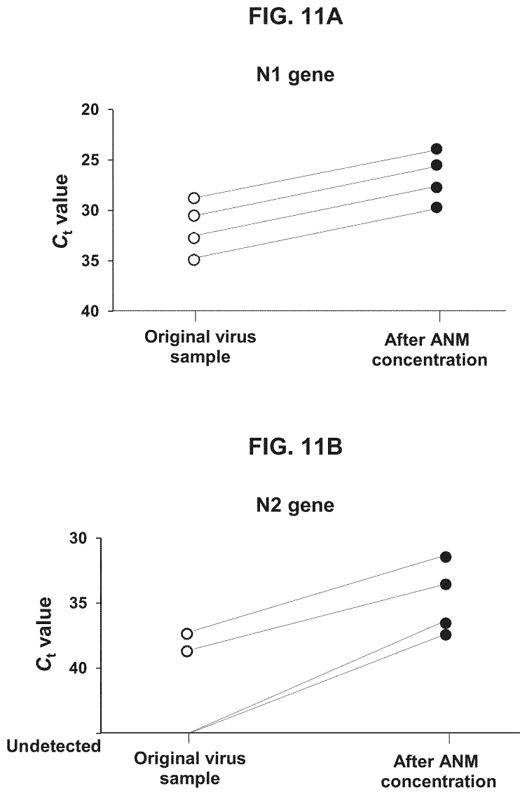

FIG. 11A-B demonstrates that the ANM device is able to enrich SARS-CoV-2 in samples with a very low viral titer (Cₜ>30) or undetectable viral titers such as when N2 gene primer-probe set was used. These results indicate that concentrating virus from the viral transport medium samples substantially improves the detection of SARS-CoV-2 in low viral load samples as compared to those same samples without ANM enrichment (average improvement in Cₜ value when using ANM devices for low viral titer samples was 5.0, n=12). These improvements in Cₜ value are consistent with results using samples with higher viral titer, indicating that the ANM devices are able to maintain a high recovery rate even for low viral load samples.

### Example 9

### ANMs Can be Operated in a Tangential-Flow Format to Allow High Throughput Purification of Inactivated or Attenuated Viruses for Vaccine-Related Application

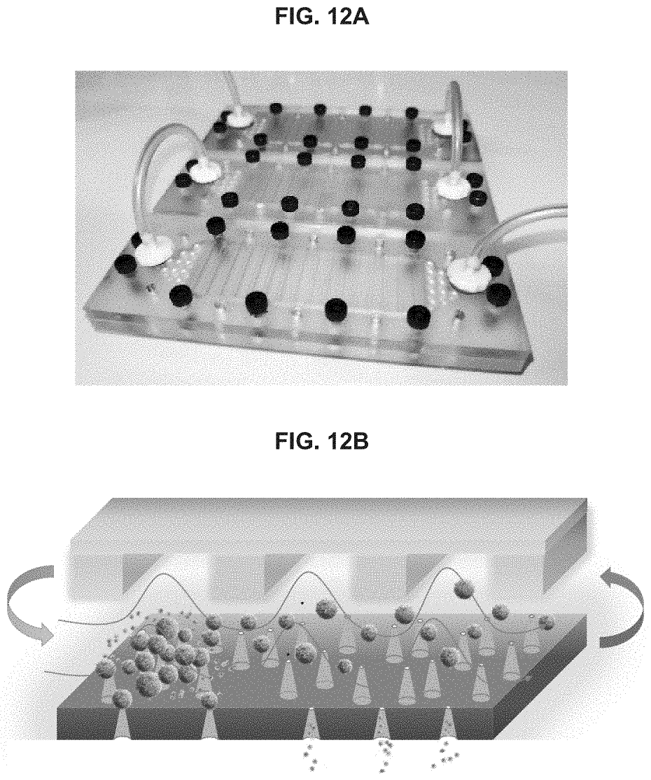

FIG. 12 demonstrates how the ANM device can be configured into a tangential flow format to allow high throughput (>40 mL/hour per chip) and scalable (multiple chips in parallel or in series) purification of virus vaccine solutions. A baffled design prevents the highly concentrated virus solution from forming a filter cake to reduce the throughput. The virus vaccines grown in cell culture reactors are contaminated by proteins that must be removed before injection. The ANM device enables this application.

1. A system for isolating viral particles comprising:
a first chamber;
a second chamber;
a membrane positioned between the first and second chambers, and comprising a first membrane surface facing and at least partially defining the first chamber, a second membrane surface facing and at least partially defining the second chamber and a plurality of asymmetrically shaped nanopores extending between the first and second membrane surfaces, wherein each nanopore includes a first nanopore opening at the first membrane surface having a first diameter, and a second nanopore opening at the second membrane surface having a second diameter that is greater than the first diameter;
a sample comprising the viral particles positioned within the first chamber; and
a device for inducing fluid flow through the membrane from the first chamber to the second chamber by pressure driven flow, electroosmotic flow, centrifugal force, or a combination thereof.

2. The system of claim 1, wherein the first membrane surface comprises one or more baffles.

3. A system for isolating viral particles comprising:
a first chamber;
a second chamber;
a membrane positioned between the first and second chambers, and comprising a first membrane surface facing and at least partially defining the first chamber, a second membrane surface facing and at least partially defining the second chamber and a plurality of asymmetrically shaped nanopores extending between the first and second membrane surfaces, wherein each nanopore includes a first nanopore opening at the first membrane surface having a first diameter, and a second nanopore opening at the second membrane surface having a second diameter that is greater than the first diameter;
wherein the first membrane surface comprises one or more baffles;
a sample comprising the viral particles positioned within the first chamber; and
a device for inducing fluid flow through the membrane from the first chamber to the second chamber by pressure driven flow, electroosmotic flow, centrifugal force, or a combination thereof.

4. The system of claim 1, wherein the first membrane surface is coated with a magnetic alloy selected from nickel-iron, samanum-cobalt, aluminum-nickel-cobalt, nickel-iron-chromium, iron-chromium-cobalt, or neodymium-iron-boron.

5. The system of claim 1, wherein the first diameter is from about 10 nm to about 200 nm.

6. The system of claim 1, wherein the first diameter of the plurality of asymmetrically shaped nanopores has a coefficient of variation of less than 10% between each nanopore.

7. The system of claim 1, wherein the second diameter is from about 30 nm to about 10 μm.

8. The system of claim 1, wherein a distance between the first and second membrane surfaces is from about 1 μm to about 100 μm.

9. The system of claim 1, wherein the membrane comprises a nanopore density from about 106 to about 1010 nanopores/cm2.

10. The system of claim 1, wherein the nanopores of the membrane are ion-etched.

11. The system of claim 1, wherein the first chamber comprises a plurality of inlets.

12. The system of claim 1, wherein the first chamber comprises a first inlet for loading of the sample into the first chamber; and,
a second inlet for loading of an elution buffer, lysing solution, PCR cocktail, or a combination thereof into the first chamber; and,
wherein a concentrated virus solution is eluted from the first chamber through the first inlet or the second inlet into a collection tube or a third chamber.

13. The system of claim 12, wherein the first inlet and second inlet are the same inlet.

14. The system of claim 1, wherein the second chamber comprises an outlet wherein the device for inducing fluid flow through the membrane from the first chamber to the second chamber is connected.

15. (canceled)

16. The system of claim 1, further comprising a fourth chamber and a filter positioned between the fourth chamber and the first chamber, the filter comprising a first filter surface facing and at least partially defining the fourth chamber, a second filter surface facing and at least partially defining the first chamber and a plurality of filter pores extending between the first and second filter surfaces, wherein each filter pore has a diameter of about 200 nm to about 5 microns.

17. (canceled)

18. The system of claim 16, wherein the membrane and filter are formed from one or more materials comprising a polyethylene terephthalate (PET), a polycarbonate (PC), a polypropylene (PP), a polyimide (PI), or a polyethersulphone (PES).

19. The system of claim 1, wherein the device for inducing fluid flow generates a flow rate of about 0.01 mL/hour to about 100 mL/hour and a pressure less than about 1 atm, and comprises a syringe pump, and electroosmotic pump, a micropump, a centrifuge, a vacutainer, a snap lock syringe pump, or a combination thereof.

20-21. (canceled)

22. The system of claim 16, wherein the sample is applied perpendicularly or tangentially to the membrane or the filter and has a flow rate of about 5 mL/hour to about 40 mL/hour.

23. (canceled)

24. The system of claim 1, wherein the viral particles are about 80-100 nm in size.

25. The system of claim 1, wherein the viral particles are SARS-COV-2 viral particles.

26. (canceled)

27. The system of claim 4, wherein the viral particles are bound to an antibody probe that is coupled to a magnetic bead.

28. (canceled)

29. The system of claim 1, wherein the system is connected with a plurality of identical systems in series or in parallel.

30-42. (canceled)


SEQUENCE LISTING (7 sequences)

SEQ ID NO: 1 - DNA, 20 bp, Artificial Sequence
  Synthetic
gaccccaaaatcagcgaaat

SEQ ID NO: 2 - DNA, 24 bp, Artificial Sequence
  Synthetic
tctggttactgccagttgaatctg

SEQ ID NO: 3 - DNA, 24 bp, Artificial Sequence
  Synthetic
accccgcattacgtttggtggacc

SEQ ID NO: 4 - DNA, 20 bp, Artificial Sequence
  Synthetic
ttacaaacattggccgcaaa

SEQ ID NO: 5 - DNA, 18 bp, Artificial Sequence
  Synthetic
gcgcgacattccgaagaa

SEQ ID NO: 6 - DNA, 23 bp, Artificial Sequence
  Synthetic
acaatttgcccccagcgcttcag

SEQ ID NO: 7 - DNA, 29882 bp, SARS-CoV-2/human/USA/WA-CDC-WA1/2020
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactttttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgttcaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaa